(19) United States
(12) Patent Application Publication
(10) Pub. No.: US 2023/0404802 A1
(43) Pub. Date: Dec. 21, 2023

(54) Dispenser for Administering Eye Drops

(71) Applicant: United State Government as Represented by the Department of Veterans Affairs, Washington, DC (US)

(72) Inventor: Julie Schallhorn, San Francisco, CA (US)

(21) Appl. No.: 18/333,978

(22) Filed: Jun. 13, 2023

Related U.S. Application Data
(60) Provisional application No. 63/351,638, filed on Jun. 13, 2022.

(51) Int. Cl. A61F 9/00

(52) U.S. Cl. CPC A61F 9/0026

(57) ABSTRACT

A dispenser for use with a squeezable eyedropper bottle containing medication is disclosed having a first arm defining a first pivot end, a first dispensing end, a first exterior surface and a first interior surface. The dispenser includes a retention feature positioned on the first arm to removably engage the squeezable eyedropper bottle. The dispenser includes a second arm defining a second pivot end, a second dispensing end, a second exterior surface and a second interior surface, the second pivot end coupled to the first pivot end of the first arm so that the first interior surface is movable relative to the second interior surface of the second arm, the second arm configured to squeeze the squeezable eyedropper bottle as the second interior surface is moved towards the first interior surface such that the medication is dispensed from the squeezable eyedropper bottle.

### CROSS-REFERENCE TO RELATED APPLICATIONS

This application claims priority to and the benefit of U.S. Provisional Patent Application No. 63/351,638, filed Jun. 13, 2022, the entirety of which is hereby incorporated by reference herein.

### FIELD

This disclosure relates to a dispenser to assist in the administration of eye drops. In particular, this disclosure relates to a dispenser for use with single use vials of eye medication that simplifies application for patients with dexterity issues or motor challenges.

### BACKGROUND

The present disclosure describes a dispenser to facilitate the application of eye drops in individuals having physical challenges. Most individuals have some degree of challenge in the application of eye drops. The unintended blink or the misdirected drop that misses they eye can require multiple attempts to properly administer the medicine. While this may be acceptable for medicines that are provided in bulk containers where multiple treatments are contained, other eye drop medication comes in single use vials where the quantity of medicine contained therein is not sufficient to sustain multiple attempts at application. In addition, the size of these vials produces additional challenges to those with physical challenges.

One such medicine is preservative-free ophthalmic medication. This medication typically is provided in single use vials that are small and difficult to manipulate for patients with dexterity issues or arthritis. Unfortunately, these patients are often the ones that require preservative-free drops. It would, therefore, be beneficial to have a device to assist in the application of vials of preservative-free ophthalmic medication that simplified and improved medication delivery for individuals with physical challenges. It would further be beneficial to have a device designed to hold the smaller preservative-free dropper and assist the patient with aligning it over the eye and administering it. This would allow patients with physical challenges to apply preservative-free ophthalmic medication safely and effectively.

### SUMMARY

Disclosed herein, in various aspects, is a dispenser for use with a squeezable eyedropper bottle containing medication. The dispenser can include a first arm, a retention feature, and a second arm. The first arm can define a first pivot end, a first dispensing end, a first exterior surface, and a first interior surface. The retention feature can be positioned on the first arm. In use, the retention feature can be configured to removably engage the squeezable eyedropper bottle. The second arm can define a second pivot end, a second dispensing end, a second exterior surface, and a second interior surface. The second pivot end can be coupled to the first pivot end of the first arm so that the first interior surface is movable relative to the second interior surface of the second arm. In use, the second arm can be configured to squeeze the squeezable eyedropper bottle as the second interior surface is moved towards the first interior surface such that the medication is dispensed from the squeezable eyedropper bottle.

In further aspects, disclosed herein is a dispensing system for eye drop medication. The system can include a squeezable eyedropper bottle and a dispenser. The squeezable eyedropper bottle can house medication. The dispenser can include a first arm, a retention feature, and a second arm. The first arm can define a first pivot end, a first dispensing end, a first exterior surface, and a first interior surface. The retention feature can be positioned on the first arm. In use, the retention feature can be configured to removably engage the squeezable eyedropper bottle. The second arm can define a second pivot end, a second dispensing end, a second exterior surface, and a second interior surface. The second pivot end can be coupled to the first pivot end of the first arm so that the first interior surface is movable relative to the second interior surface of the second arm. In use, the second arm can be configured to squeeze the squeezable eyedropper bottle as the second interior surface is moved towards the first interior surface such that the medication is dispensed from the squeezable eyedropper bottle.

### BRIEF DESCRIPTION OF THE DRAWINGS

FIG. 1 is a perspective illustration of an exemplary dispenser according to the disclosure;

FIG. 2 is a perspective illustration of another exemplary dispenser according to the disclosure;

FIG. 3 is a side view schematic illustration of an exemplary use of the dispenser shown in FIG. 1;

FIG. 4 is a side view of the dispenser of FIG. 1, with the dispenser shown in a closed position;

FIG. 5 is an illustrative photograph of squeezable eyedropper bottles containing medication for use with a dispenser as disclosed herein;

FIG. 6 is a perspective illustration of the dispenser shown in FIG. 1, with the dispenser illustrated housing receiving a squeezable eyedropper bottle;

FIG. 7 is a side view illustration of the dispenser shown in FIG. 1, with the dispenser shown squeezing a squeezable eyedropper bottle and dispensing medication as disclosed herein;

FIG. 8 is a side perspective view of an exemplary dispenser having a latch element that retains a squeezable eyedropper bottle in a retention element as disclosed herein; and

FIG. 9 is a side perspective view of an exemplary dispenser having a clip that retains a squeezable eyedropper bottle in a retention element as disclosed herein.

### DETAILED DESCRIPTION

The present disclosure can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

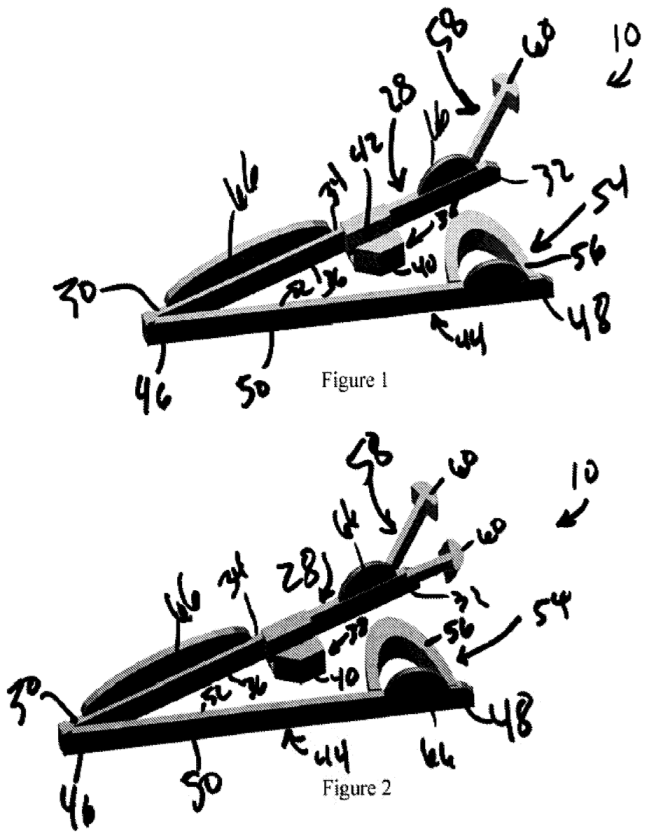

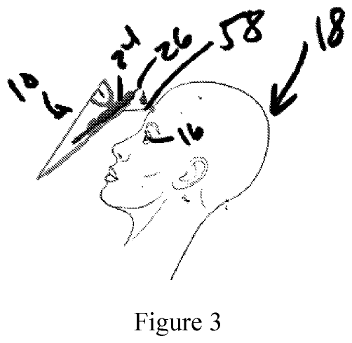

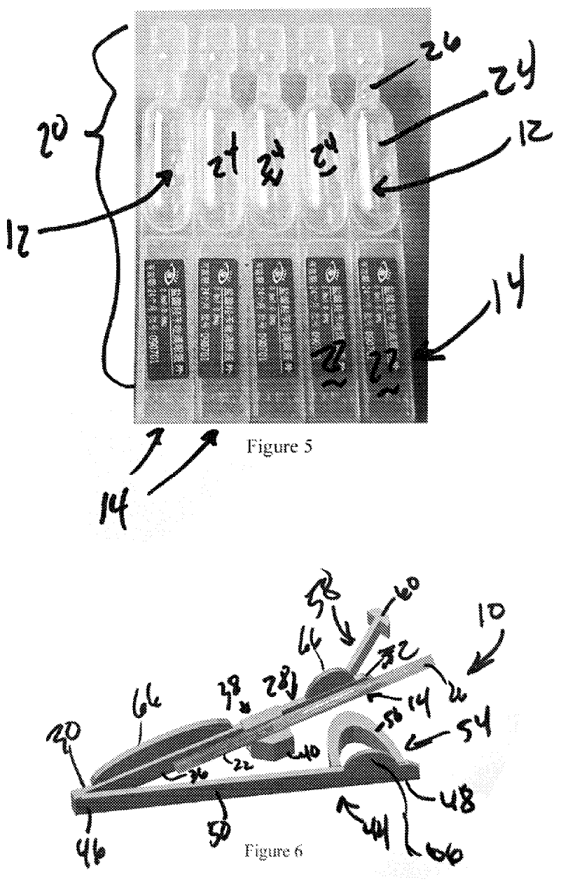

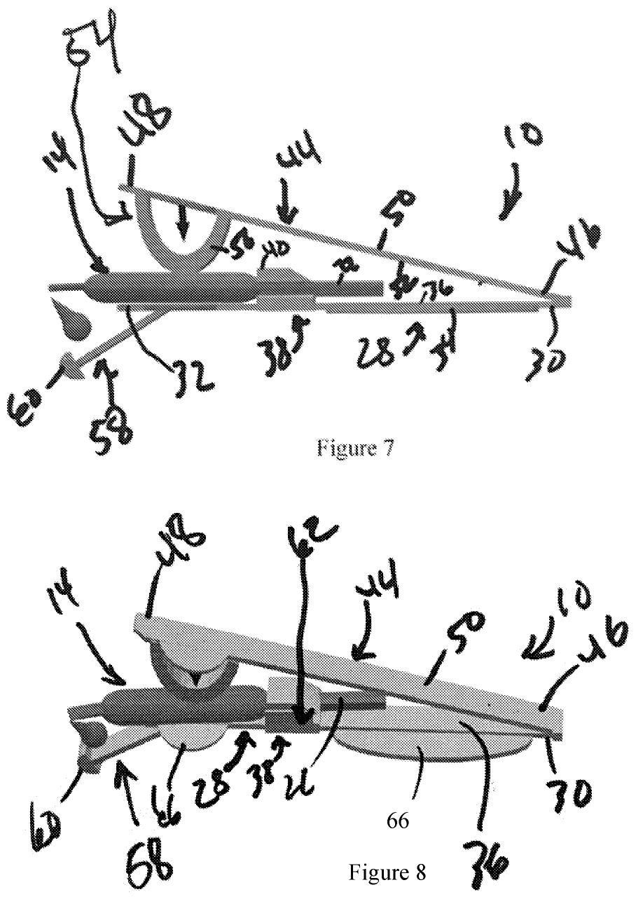

FIGS. 1-4 show an example of a dispenser 10 in accordance with one aspect of the current disclosure. The dispenser 10 is configured to house and dispense medication 12 from a squeezable eyedropper bottle 14 (see FIG. 5) into the eye 16 of a user 18 (see FIG. 3). In one aspect of the disclosure the squeezable eyedropper bottle 14 comprises a vial 20. In particular, in one aspect of the disclosure the squeezable eyedropper bottle 20 comprises a flattened base section 22, a flexible bulbous storage portion 24 and a dispensing nozzle 26 (see FIG. 5). In one aspect of the disclosure the medication 12 may comprise preservative-free ophthalmic medication. In other aspects, a wide range of squeezable eyedropper bottles 14 and medications 12 are contemplated. The dispenser 10, in one aspect, includes a first arm 28. The first arm 28 defines a first pivot end 30, a first dispensing end 32, a first exterior surface 34 and a first interior surface 36. In one aspect the dispenser 10, the first arm 28 and the remaining disclosure features are formed using 3D printing. In another aspect, the dispenser 10, the first arm 28, and the remaining disclosure features are formed using injection molding. In still another aspect, the dispenser 10 may be formed using known manufacturing techniques.

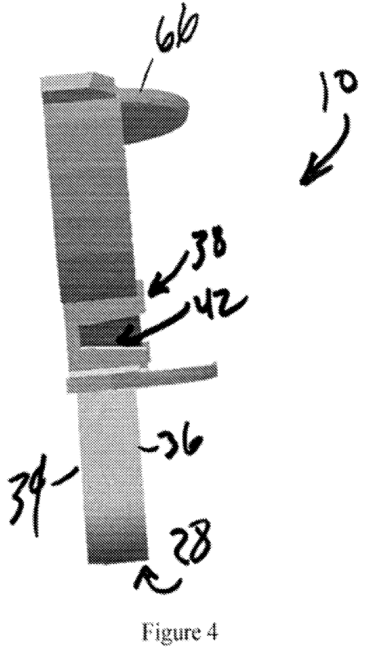

The dispenser 10 may further include a retention feature 38 positioned on the first arm 28. The retention feature 38 is configured to removably engage one of the squeezable eyedropper bottles 14 (FIG. 1-3). The retention feature 38 securely holds the squeezable eyedropper bottle 14 in a position parallel to the first arm 28. In one aspect the retention feature 38 may comprise a projection 40 from the first interior surface 36. In one aspect, the retention feature 38 may be molded as a unitary feature with the first arm 28. In another aspect, the retention feature 38 may be mounted to the first arm 28. As shown in FIG. 3, the retention feature 38 may further include a tapered slot 42 formed within the projection 40. In one exemplary aspect, the tapered slot 42 may be configured to frictionally engage the flattened base section 22 of the squeezable eyedropper bottle 14. This allows the squeezable eyedropper bottle 14 to be retained in the tapered slot 42 during operation of the dispenser 10 (see FIG. 4).

The dispenser 10 may also include a second arm 44 which defines a second pivot end 46, a second dispensing end 48, a second exterior surface 50 and a second interior surface 52. The second pivot end 46 is coupled to the first pivot end 30 of the first arm 28 so that the first interior surface 36 is movable relative to the second interior surface 52 of the second arm 44. In one aspect of the disclosure, the movement of the first interior surface 36 is movable relative to the second interior surface 52 may be achieved through a pivot hinge. In another aspect of the disclosure, the movement of the first interior surface 36 is movable relative to the second interior surface 52 may be achieved through a living hinge. In still another aspect of the disclosure, the movement of the first interior surface 36 is movable relative to the second interior surface 52 may be achieved through a fixed joint and accomplished through the flexibility of either the first arm 28, the second arm 44 or both. For example, the first arm 28 and/or the second arm 44 can resiliently deflect to effect movement of the first interior surface 36 toward the second interior surface 52 The second arm 44 is configured to squeeze the squeezable eyedropper bottle 14 as the second interior surface 52 is manually moved toward the first interior surface 36. In this fashion, the squeezable eyedropper bottle 14, and in one particular aspect the flexible bulbous section 24 is compressed and the medication 12 is forced out through the dispensing nozzle 26 (see FIGS. 3 and 5).

Although it is contemplated that the second arm 44 may be configured to squeeze the squeezable eyedropper bottle 14 as the second interior surface 52 is manually moved toward the first interior surface 36, in one particular aspect of the disclosure the dispenser 10 includes a pressure protrusion 54 extending from the second interior surface 52 (see FIG. 1). The pressure protrusion 54 is sized such that connection and pressure to the flexible bulbous section 24 is achieved prior to the second interior surface 52 making contact with any portion of the first interior surface 36 or the retention feature 38. In one particular aspect, the pressure protrusion 54 comprises an arched arm 56 protruding from the second interior surface 52. The arched arm 56 allows for sufficient strength to compress the flexible bulbous section 24 while allowing the size and weight of the dispenser 10 to be minimized.

The dispenser 10, in another aspect, may include at least one forehead/eyelid engagement arm 58 (see FIG. 1). The forehead/eyelid engagement arm 58 allows the user of the dispenser 10 to properly position the dispenser 10 over their eye 16 while accommodating instability in hand movements (see FIG. 3). In addition, the stabilization provided by the forehead/eyelid engagement arm 58 allows for both hands to operate the moving of the second arm 44 toward the first arm 28 for users that do not have the strength for single hand operation. The forehead/eyelid engagement arm 58, in one aspect, may include a curved forehead/eyelid engagement surface 60 that provides a comfortable and non-invasive contact with the user's forehead/eyelid. In one aspect, the forehead/eyelid engagement arm 58 engages the forehead to insure stability during dispensing of the medication 12. In another aspect, the forehead/eyelid engagement arm 58 engages the eyelid of the subject to both provide stability as well as prevent unwanted blinking during application. In still another aspect of the disclosure, the forehead/eyelid engagement arm 58 may comprise a plurality of forehead/eyelid engagement arms 58 (see FIG. 2). In this aspect, the duality of the engagement arms 58 may provide additional stabilization or a combination of stabilization and eyelid blinking prevention. Although a variety of positions are contemplated for the forehead/eyelid engagement arm 58 relative to the first arm 28, in at least one aspect of the disclosure the forehead/eyelid engagement arm 58 is angled between 20 and 45 degrees from the first arm 28. The angle of the forehead/eyelid engagement arm 58 to the first arm 28 helps improve medication application direction relative to the user's placement of the engagement arm 58. In this fashion, a user may find a physical feature on their forehead (such as an eyebrow) and have the security that the medication 12 will be properly applied.

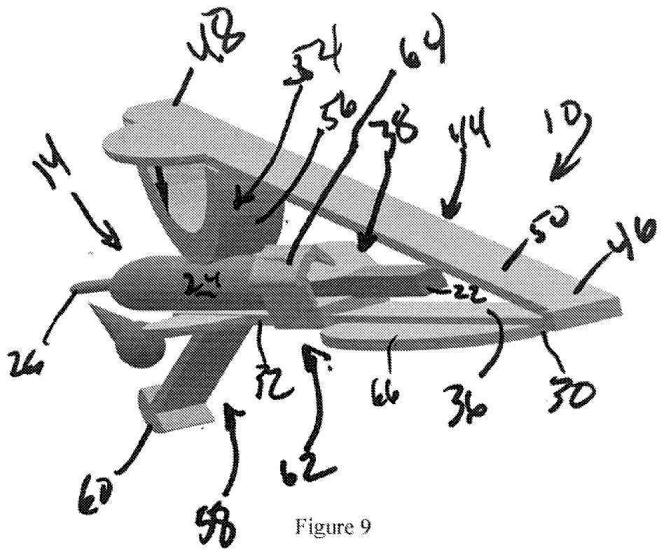

Referring now to FIGS. 8 and 9, the dispenser 10 may further include a latch element 62 mounted or formed into the retention feature 38. The latch element 62, in one aspect, comprises a flexible cover over the tapered slot 42 that additionally secures the squeezable eyedropper bottle 14 within the retention feature 38. The latch element 62 allows the retention feature 38 to accommodate a wider range of squeezable eyedropper bottles 14 from different manufacturers or of different sizes while still allowing the user to securely bring the medication 12 to their eye 16. In another aspect, the latch element 62 may comprise a clip 64 (see FIG. 9) that provides additional security to the squeezable eyedropper bottle 14 while the user raises it to their eye 16.

In one additional aspect of the disclosure, the dispenser 10 may include a plurality of stiffening elements 66 formed or mounted on the first arm 28 and/or the second arm 44. The stiffening elements 66 on the first arm 28 allow the first arm 28, retaining the squeezable eyedropper bottle 14 to remain in a stable position. The stiffening elements 66 mounted on the second arm 44 allow the second arm 44 to flex in such a way that the pressure protrusion 54 properly engages the flexible bulbous section 24. This allows a single molded or printed dispenser 10 to provide all the characteristics described in the present disclosure.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.
Aspect 1: A dispenser for use with a squeezable eyedropper bottle containing medication, the dispenser comprising:
a first arm defining a first pivot end, a first dispensing end, a first exterior surface and a first interior surface;
a retention feature positioned on the first arm, the retention feature configured to removably engage the squeezable eyedropper bottle; and
a second arm defining a second pivot end, a second dispensing end, a second exterior surface and a second interior surface, the second pivot end coupled to the first pivot end of the first arm so that the first interior surface is movable relative to the second interior surface of the second arm, the second arm configured to squeeze the squeezable eyedropper bottle as the second interior surface is moved towards the first interior surface such that the medication is dispensed from the squeezable eyedropper bottle.
Aspect 2: The dispenser of aspect 1, wherein the retention feature is molded on the first interior surface.
Aspect 3: The dispenser of aspect 1, wherein the retention feature is mounted on the first interior surface.
Aspect 4: The dispenser of aspect 1, wherein the retention feature comprises a projection from the first interior surface, the projection having a tapered slot formed therein.
Aspect 5: The dispenser of aspect 1, further comprising a latch element mounted on the retention feature.
Aspect 6: The dispenser of aspect 5, wherein the latch element is rotatably mounted to the retention feature.
Aspect 7: The dispenser of aspect 5, wherein the latch element comprises a clip.
Aspect 8: The dispenser of aspect 1, further comprising:
at least one forehead engagement arm extending from the first exterior surface.
Aspect 9: The dispenser of aspect 8, wherein the at least one forehead engagement arm comprises a curved forehead engagement surface.
Aspect 10: The dispenser of aspect 8, wherein the at least one forehead engagement arm extends from the first exterior surface at an angle between 20 degrees and 45 degrees.
Aspect 11: The dispenser of aspect 8, wherein the at least one forehead engagement arm comprises a plurality of forehead engagement arms.
Aspect 12: The dispenser of aspect 8, further comprising:
a pressure protrusion extending from the second interior surface, the pressure protrusion configured to engage the squeezable eyedropper bottle when the second interior surface is moved towards the first interior surface.
Aspect 13: The dispenser of aspect 12, wherein the pressure protrusion comprises an arched arm protruding from the second interior surface.
Aspect 14: The dispenser of aspect 1, further comprising a simple hinge that couples the second pivot end to the first pivot end.
Aspect 15: A dispensing system for eye drop medication, comprising:
a squeezable eyedropper bottle housing medication; and
a dispenser comprising:
   a first arm defining a first pivot end, a first dispensing end, a first exterior surface and a first interior surface;
   a retention feature positioned on the first arm, the squeezable eyedropper bottle mounted within the retention feature; and
   a second arm defining a second pivot end, a second dispensing end, a second exterior surface and a second interior surface, the second pivot end coupled to the first pivot end of the first arm so that the first interior surface is movable relative to the second interior surface of the second arm, the second arm configured to squeeze the squeezable eyedropper bottle as the second interior surface is moved towards the first interior surface such that the medication is dispensed from the squeezable eyedropper bottle.
Aspect 16: The dispensing system of aspect 15, wherein the retention feature is molded on the first interior surface.
Aspect 17: The dispensing system of aspect 16, wherein the retention feature comprises a projection from the first interior surface, the projection having a tapered slot formed therein.
Aspect 18: The dispensing system of aspect 16, further comprising a latch element rotatably molded to the retention feature.
Aspect 19: The dispensing system of aspect 18, wherein the latch element comprises a clip.
Aspect 20: The dispensing system of aspect 15, further comprising:
at least one forehead engagement arm extending from the first exterior surface.
Aspect 21: The dispensing system of aspect 20, wherein the at least one forehead engagement arm is removably mounted to the first exterior surface.
Aspect 22: The dispensing system of aspect 20, wherein the at least one forehead engagement arm comprises a curved forehead engagement surface.
Aspect 23: The dispensing system of aspect 20, wherein the at least one forehead engagement arm extends from the first exterior surface at an angle between 20 degrees and 45 degrees.
Aspect 24: The dispensing system of aspect 20, wherein the at least one forehead engagement arm comprises a plurality of forehead engagement arms.
Aspect 25: The dispensing system of aspect 15, further comprising:
a pressure protrusion extending from the second interior surface, the pressure protrusion configured to engage the squeezable eyedropper bottle when the second interior surface is moved towards the first interior surface.
Aspect 26: The dispensing system of aspect 25, wherein the pressure protrusion comprises an arched arm protruding from the second interior surface.
Aspect 27: The dispensing system of aspect 15, further comprising a simple hinge that couples the second pivot end to the first pivot end.
Aspect 28: A method of dispensing medication from a squeezable eyedropper bottle comprising:
placing the squeezable eyedropper bottle within a retention feature of a dispenser, the dispenser comprising a first arm defining the retention feature and a second arm pivotably connected to the first arm;
squeezing the first arm and the second arm together such that the squeezable eyedropper bottle is compressed and the medication is dispensed.
Aspect 29: The method of aspect 28, further comprising:
placing a forehead engagement arm against a forehead prior to squeezing the first arm and the second arm.

Those skilled in the art will recognize, or be able to ascertain using no more than routine experimentation, many equivalents to the specific embodiments of the method and compositions described herein. Such equivalents are intended to be encompassed by the following claims.

What is claimed is:

1. A dispenser for use with a squeezable eyedropper bottle containing medication, the dispenser comprising:
a first arm defining a first pivot end, a first dispensing end, a first exterior surface and a first interior surface;
a retention feature positioned on the first arm, the retention feature configured to removably engage the squeezable eyedropper bottle; and
a second arm defining a second pivot end, a second dispensing end, a second exterior surface and a second interior surface, the second pivot end coupled to the first pivot end of the first arm so that the first interior surface is movable relative to the second interior surface of the second arm, the second arm configured to squeeze the squeezable eyedropper bottle as the second interior surface is moved towards the first interior surface such that the medication is dispensed from the squeezable eyedropper bottle.

2. The dispenser of claim 1, wherein the retention feature is molded on the first interior surface.

3. The dispenser of claim 1, wherein the retention feature is mounted on the first interior surface.

4. The dispenser of claim 1, wherein the retention feature comprises a projection from the first interior surface, the projection having a tapered slot formed therein.

5. The dispenser of claim 1, further comprising a latch element mounted on the retention feature.

6. The dispenser of claim 5, wherein the latch element is rotatably mounted to the retention feature.

7. The dispenser of claim 5, wherein the latch element comprises a clip.

8. The dispenser of claim 1, further comprising:
at least one forehead engagement arm extending from the first exterior surface.

9. The dispenser of claim 8, wherein the at least one forehead engagement arm comprises a curved forehead engagement surface.

10. The dispenser of claim 8, wherein the at least one forehead engagement arm extends from the first exterior surface at an angle between 20 degrees and 45 degrees.

11. The dispenser of claim 8, wherein the at least one forehead engagement arm comprises a plurality of forehead engagement arms.

12. The dispenser of claim 8, further comprising:
a pressure protrusion extending from the second interior surface, the pressure protrusion configured to engage the squeezable eyedropper bottle when the second interior surface is moved towards the first interior surface.

13. The dispenser of claim 12, wherein the pressure protrusion comprises an arched arm protruding from the second interior surface.

14. A dispensing system for eye drop medication comprising:
a squeezable eyedropper bottle housing medication; and
a dispenser comprising:
a first arm defining a first pivot end, a first dispensing end, a first exterior surface and a first interior surface;
a retention feature positioned on the first arm, the squeezable eyedropper bottle mounted within the retention feature; and
a second arm defining a second pivot end, a second dispensing end, a second exterior surface and a second interior surface, the second pivot end coupled to the first pivot end of the first arm so that the first interior surface is movable relative to the second interior surface of the second arm, the second arm configured to squeeze the squeezable eyedropper bottle as the second interior surface is moved towards the first interior surface such that the medication is dispensed from the squeezable eyedropper bottle.

15. The dispensing system of claim 14, wherein the retention feature is molded on the first interior surface.

16. The dispensing system of claim 15, wherein the retention feature comprises a projection from the first interior surface, the projection having a tapered slot formed therein.

17. The dispensing system of claim 14, further comprising:
at least one forehead engagement arm extending from the first exterior surface.

18. The dispensing system of claim 17, wherein the at least one forehead engagement arm comprises a plurality of forehead engagement arms.

19. A method of dispensing medication from a squeezable eyedropper bottle, the method comprising:
placing the squeezable eyedropper bottle within a retention feature of a dispenser, the dispenser comprising a first arm defining the retention feature and a second arm pivotably connected to the first arm; and
squeezing the first arm and the second arm together such that the squeezable eyedropper bottle is compressed and the medication is dispensed.

20. The method of claim 19, further comprising:
placing a forehead engagement arm against a forehead prior to squeezing the first arm and the second arm.
